(19) United States
(12) Patent Application Publication
(10) Pub. No.: US 2010/0004525 A1
(43) Pub. Date: Jan. 7, 2010

(54) MEDICAL CATHETER WITH SEVERAL POLES OR ELECTRODES

(76) Inventor: Peter Osypka, Rheinfelden-Herten (DE)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 12/497,085

(22) Filed: Jul. 2, 2009

(30) Foreign Application Priority Data
Jul. 5, 2008 (DE) 102008032500.7

(51) Int. Cl. A61B 5/04; A61N 1/05

(52) U.S. Cl. 600/373; 607/116

(57) ABSTRACT

A medical catheter (1) that has, on its distal end (2), several poles (3) or electrodes that are arranged axially one next to the other and that are used for stimulating and/or detecting potentials from an organ of the human body. A coil made from as many parallel running, electrically conductive windings as there are poles (3) is provided. The windings are bare, so that the entire cross section of the coil and thus of the catheter (1) can be kept small, and insulation (4), for example, in the form of a plastic cord, extends between the windings that form the poles (3). In this way, the outer dimensions of the catheter (1) are relatively small and, in addition, its distal end (2) has good flexibility.

### CROSS-REFERENCE TO RELATED APPLICATIONS

This application claims the benefit of German Application No. DE 10 2008 032 500.7, filed Jul. 5, 2008, which is incorporated herein by reference as if fully set forth.

### BACKGROUND

The invention relates to a medical catheter with several poles or electrodes arranged axially one next to the other or one behind the other for stimulating and/or for detecting potentials from an organ of the human body, for example, from the heart, from the brain, or from the ear, with a coil formed from several electrically conductive windings running parallel to each other, wherein each winding leads to a pole arranged at the distal end of the catheter.

From DE 44 25 195 C1, a medical catheter of this type with several poles or electrodes is known. The electrodes arranged axially one behind the other are here arranged on an insulated metal coil that is stripped of insulation at certain points on the distal end. This, however, produces poles with only a relatively small surface area, so that, under some circumstances, the contact could be endangered due to anatomical deviations.

In a bipolar electrode known from practice, a coil of smaller cross section is provided for one pole and a coil of larger cross section is provided for the other pole, so that a coaxial and concentric arrangement of these coils is required to obtain merely two poles.

### SUMMARY

Therefore, there is the objective of creating a catheter of the type noted above in which several poles could be arranged with an essentially matching extent, thus coils of different diameters are avoided, wherein, nevertheless, each pole should be able to have relatively the same size surface area.

To meet this objective, the catheter according to the invention has windings for forming the poles at the distal end of the catheter which are bare windings, not to be stripped, and insulation is arranged between the bare windings at least in the area of the poles, wherein this insulation is formed, on its side, by windings that run between the bare windings and that are made from insulating material or by wound plastic cords.

In this way, poles are made available that can reach across the entire extent of the catheter, wherein it is even possible that such a pole also reaches across several windings, that is, could have a large extent. The windings running between the bare windings and made from insulating material or the wound plastic cords arranged there produce an especially simple type of insulation through which it could be guaranteed that the bare windings extending in parallel one next to the other for the different poles can not come into electrical contact with each other.

It is further advantageous that a large number of different poles are provided, wherein, nevertheless, the production is simple, because the lines and wires leading to the poles are constructed as bare windings or windings that are bare at least in the pole region and wound insulation runs between these bare windings and the wires do not have to be stripped.

For a selected arrangement of the individual poles, it can be preferable when, for forming the poles by the windings, at least the last winding turn at the distal end is left bare at least partially or over the entire extent of the coil or when the windings for forming the poles are left bare over a fraction of the length or over the entire length of the coil. Therefore, the poles arranged one next to the other on the several windings are arranged at the distal end of the catheter and can be used there effectively with their relatively large surface area.

Leaving the windings bare over the entire length of the coil is possible by the measure according to the invention, wherein insulation is arranged between the windings leading to different poles, so that insulation for the windings of the electrical conductors or wires forming the coils themselves is unnecessary.

The wound insulation arranged between the electrically conductive windings can have a cross-sectional shape that is round or that is adapted to the shape of the intermediate spaces between the windings forming the poles. Thus, an essentially smooth outer side of the catheter could be formed in which, for example, round wires form the electrically conductive windings and the intermediate spaces located between the wires due to the round cross section of these wires could be filled with a corresponding negative cross section.

It is preferable when windings are formed from wires, multi-strand wires, electrically conductive flat wires, and/or round wires and are shaped into a multi-turn coil. In this way, the catheter can be adapted to anatomical requirements.

Another construction provides that the windings are coated or covered with insulation in the direction toward the proximal end of the catheter such that the poles do not repeat on a meridian line of the coils in the direction toward the proximal end. For example, an outer coating could reach over the coil formed from several parallel windings up to the distal end region in which the individual poles should be active and this area is left open accordingly.

Thus, the catheter can have an outer, insulating coating that reaches up to the distal end with the poles and that has openings in the area of the bare windings or wires or conductors for forming poles with small surface areas. In this way, the coating could have an opening for each pole in the region of the windings that are left bare and that form the poles on the periphery, and these openings could advantageously be arranged coaxially one next to the other.

Another advantageous construction of the invention provides that the number of poles is increased so that, in the inner lumen of the windings of the coil forming the first poles, conductors or wires are guided beyond the distal end of the coil to a carrier with elements that form additional poles and that are each connected to one of the conductors. Thus, the inner lumen of the coil that essentially forms the catheter and that is enclosed by the individual windings could also be used to form additional poles, but without having to increase the cross section of the catheter.

Here it is advantageous when the additional poles are arranged one next to the other on the carrier in the axial direction of the catheter. In this way they could be set either in line one next to the other or optionally offset relative to each other on the periphery.

The carrier can carry contact points arranged on its outside or peripheral rings that are bare at least in some regions and that serve as additional poles and that are connected to the conductors guided through the inner lumen of the coil. The conductors could thus be introduced practically from the inside into the carrier and then connected to the contact points or rings located on the outside.

At least one part or all of the conductors guided through the inner lumen of the coil to additional poles provided on the distal end of the catheter could be insulated, so that opposing electrical contacts are avoided.

Another construction of the catheter according to the invention could provide that its distal end having the poles has a decreasing or tapering, in particular, conical shape at least over one part of its axial extent. This simplifies introducing the catheter into openings or cavities of a body and also provides good effectiveness above all in narrow vessels or cavities.

The inner lumen of the coil essentially forming the catheter can have an open cross section, such that there is space for a stiletto or a mandrin or similar guide element with or without additional conductors running therein. In this way, the handling of the catheter could be simplified.

Another construction of the catheter according to the invention provides that it has an outer, insulating coating that has openings in the region of the bare windings or wires for forming small surface area poles. Thus, through an insulating coating, its extent on the periphery of the catheter could be preset even in the region of the poles and reduced relative to the total periphery, wherein simultaneously the position of the individual poles could also be preset through such openings in an outer coating.

In this way, on the periphery the coating could have only one opening in the region of the bare windings for forming each of the poles and these openings could advantageously be arranged coaxially one next to the other. Thus, all of the poles lie in a line one next to the other. Applications are also conceivable, however, in which the individual poles left free by the coating are distributed relative to each other on the periphery of the catheter.

Another construction of the catheter provides that, on the outside of the bare windings forming the poles, there are electrically conductive or metal rings, and insulation between these rings, wherein a winding forming one pole contacts one metal ring. In this way, the poles running at an angle due to the pitch of the individual windings could be reshaped at the distal end of the catheter above the mentioned metal rings into poles that run coaxial to the center axis of the catheter on the periphery of the distal end. In this way, a rotation of the catheter nevertheless always produces the same relative position of the corresponding region of the poles relative to a body part.

Above all, for the combination of individual or several of the features and measures described above, a medical catheter is produced in which a large number of different poles could be provided, wherein nevertheless the production is simple, because the conductors and wires leading to the poles are constructed as windings that are bare or at least bare in the pole region and insulation extends between these windings, so that the wires do not have to be stripped.

### BRIEF DESCRIPTION OF THE DRAWINGS

Embodiments of the invention will be described in greater detail below with reference to the drawing. Shown in a partially schematic diagram are:

FIG. 1 is a longitudinal section view through the distal end of a medical catheter according to the invention with several, in this case, namely sixteen poles or electrodes arranged axially one next to the other for stimulating and/or detecting potentials from an organ with a multi-turn coil formed from several electrically conductive windings extending parallel to each other, wherein each winding leads to one of the poles and the windings are bare at least at the distal end of the catheter and insulation is arranged between these windings,

FIG. 2 is a view of a partial region of the coil essentially forming the catheter with two parallel windings and a cross-sectional shape of the insulation lying in-between modified relative to FIG. 1,

FIG. 3 is a diagram according to FIG. 2, wherein the electrically conductive windings are formed by multi-strand wires,

FIG. 4 is a view of an arrangement in which the electrically conductive windings are formed by flat wires and the insulation in-between has a correspondingly adapted shape,

FIG. 5 is a longitudinal section view of a modified embodiment in which the distal end of the catheter with the individual poles also has a coating in which are arranged rings in contact with the bare coils,

FIG. 6 is a view of a similarly modified embodiment in which the coating of the coils runs up to the distal end and has, at the poles to be formed, cutouts or openings below which extend the bare windings of the individual poles,

FIG. 7 is a side view of a medical catheter according to the invention approximately according to FIG. 6 with the proximal end, wherein, however, additional poles are arranged at the distal end,

FIG. 8 is an enlarged diagram of the distal end of the catheter according to FIG. 7,

FIG. 9 is a longitudinal section of the carrier with the additional poles for the distal end of the catheter according to FIGS. 7 and 8 with conductors that run in the inner lumen of the coil forming the catheter and that extend toward metal rings forming individual poles on the outside of the carrier, and

FIG. 10 is a side view of an injection-molded part that is made from plastic and that forms the carrier for additional poles at the distal end of the catheter according to FIG. 7.

### DETAILED DESCRIPTION OF THE PREFERRED EMBODIMENT

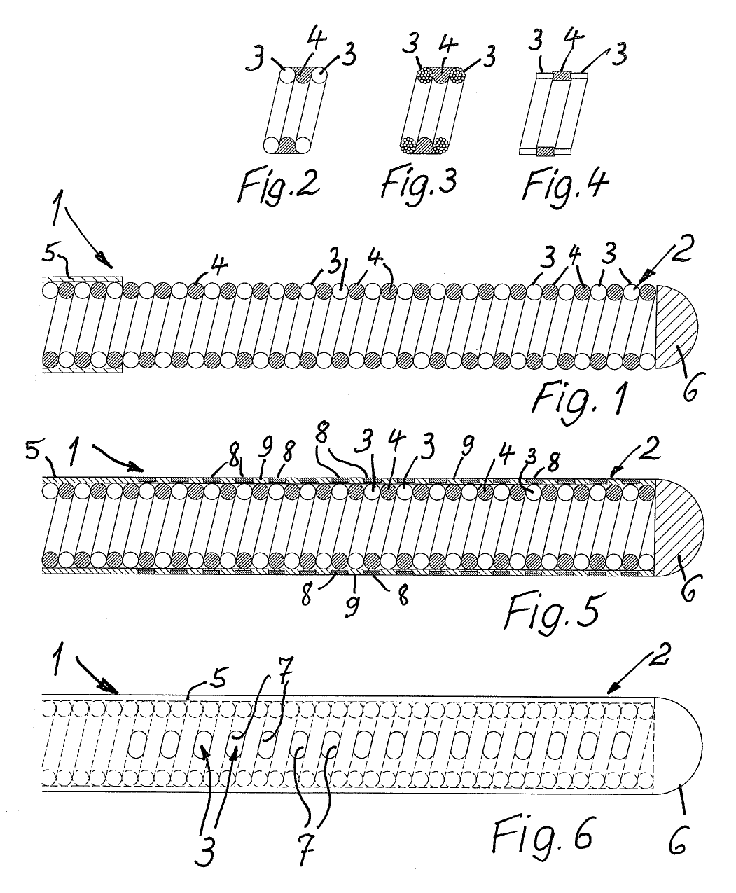

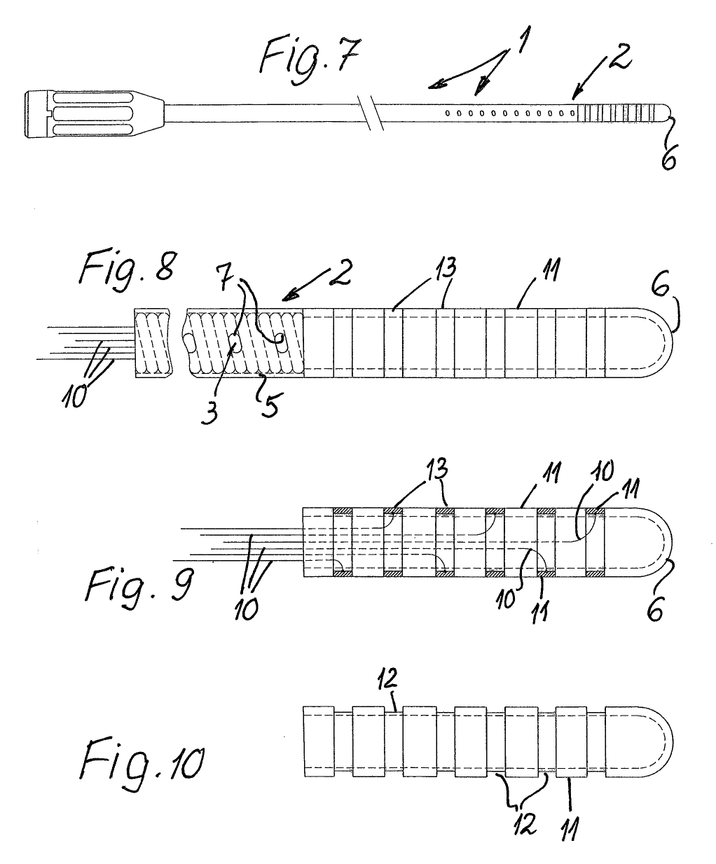

In FIGS. 1, 5, and 6, longitudinal sections of the distal end 2 of a catheter designated as a whole with 1 are shown on which, in these embodiments, a total of sixteen poles 3 are arranged axially one behind the other or one next to the other, while a modified embodiment of a catheter 1 is to be seen in FIG. 7 in an overall view.

All of the embodiments have in common that a coil formed from several parallel, electrically conductive windings is provided, wherein each winding leads to a pole 3 arranged at the distal end 2 of the catheter 1 as shown especially well in FIG. 1 but also in FIGS. 5 and 6, as well as in FIG. 8.

In this way, in all of the embodiments it is provided that the windings are bare for forming the poles 3 and that, according to FIGS. 1 to 5, insulation 4 is arranged between the windings and thus between the poles 3. The production of the coil by bare windings is simpler and allows smaller dimensions than if the windings were insulated themselves. In this way, a flexible coil that is easy to manufacture is produced that could also have a large number of poles 3 one next to the other as a medical catheter 1, because the bare conductors are separated from each other by the insulation 4 running between the conductors just as well or even better than by insulation of the windings themselves.

Here it is sufficient for an arrangement according to FIG. 1 or 5 or 6 when the last winding turn located at the distal end is bare, because, in these embodiments, only these last winding turns of the coil are provided as the sixteen poles 3.

The insulation 4 between the electrically conductive windings can be formed, on their side, by windings made from insulating material or by wound plastic cords that have a cross-sectional shape that is round (FIGS. 1 and 5) or that is adapted to the shape of the intermediate spaces between the windings forming the poles 3 (FIGS. 2, 3, and 4). In this way, poles 3 that are well insulated from each other are produced and nevertheless a distal end 2 of the catheter 1 that can have high flexibility and very small dimensions is also produced.

According to FIGS. 1 to 4, the windings forming the poles 3 could be made from wires, stranded wires (FIG. 3), flat wires (FIG. 4), or round wires (FIGS. 1, 2, and 5) and could be shaped into the mentioned multi-turn coil.

Here, one sees especially well in FIG. 1, but also in additional figures that the windings could be insulated in the direction toward the proximal end of the catheter 1 by a coating 5 at least on the outside such that the poles 3 do not repeat on a meridian line of the coil in the direction toward the proximal end. Indeed, the poles 3 are also present under the coating through the bare windings, but these cannot be active due to this coating.

In the embodiment according to FIG. 6, the coating 5 is also projected past the distal end 2, thus it reaches up to a terminal 6 arranged at this distal end and has, in the region of the last windings, openings 7 so that the bare windings located under the coating 5 could be active as poles 3 at limited points. Here, the coating 5 has, in the region of each of the bare windings forming the poles 3, only one opening 7 on the periphery, and one sees in FIG. 6 that these openings 7 could be preferably arranged axially one next to the other. Naturally, openings 7 offset on the periphery and optionally several such openings 7 belonging to each of the poles 3 would also be conceivable.

In FIG. 5 one sees an arrangement in which electrically conductive or metallic rings 8 and, between these windings, insulation 9 are arranged on the outside of the bare windings forming the poles 3, wherein a winding forming a pole 3 contacts such a metal ring 8. In this way, the poles 3 that simultaneously have an axial extent in the embodiment according to FIG. 1 in their peripheral direction because they are shaped as helical winding turns form coaxial poles directed to the outside whose periphery remains at the same axial position of the catheter 1 and thus also allows an arbitrarily rotated implantation of the catheter 1 with a constant position of the poles 3.

In FIGS. 7 to 10, improvements of a catheter 1 are shown in more detail. Here it is provided that the number of poles 3 is increased such that, in the inner lumen, that is, in the inner channel of the catheter 1 left free from the windings of the coil, conductors or wires 10 are led past the distal end of the coil to a carrier 11 that is connected to it in the coaxial direction and that has elements still to be explained, wherein these elements form additional poles 13. These elements are each connected to one of the conductors 10. Here, one sees primarily in FIGS. 7 to 9 that the additional poles 13 are arranged one next to each other and parallel to each other on the carrier 11 in the axial direction of the catheter 1.

According to FIGS. 8 and 9, the carrier 11 has contact points arranged on its outside, namely bare rings that run coaxially and concentrically and that serve as additional poles 13 and that are connected individually to the conductors 10 guided through the inner lumen of the coil, as is shown especially well in FIG. 9.

The carrier 11 is here an injection-molded part according to FIG. 10 made from plastic and has, at the position of the additional poles 13, recesses 12 that hold these ring-shaped, additional poles 13. The conductors 10 guided through the inner lumen of the coil to the additional poles 13 are insulated, which cannot lead to an increase in the cross section of the catheter 1, because they extend in the interior of the coil.

The inner lumen of the coil here already has, due to the construction of the catheter 1, a cross section such that there is sufficient place therein for a stiletto or a mandrin or similar guide element even if the additional conductors 10 are provided.

The distal end 2 with the poles 3 or 13 could have a conical shape over part of its extent. In the embodiments, however, only a rounded terminal 6 is provided that similarly simplifies the insertion of the catheter into body openings or into blood vessels.

The medical catheter 1 has, on its distal end 2, several poles 3 or electrodes that are arranged axially one next to the other and that are used for stimulating and/or detecting potentials from an organ of the human body. Here, a coil is provided that is made from as many parallel extending, electrically conductive windings as there are poles 3. The windings are bare, so that the total cross section of the coil and thus the catheter 1 can be kept small and insulation 4 runs between the windings that form the poles 3, for example, in the form of a plastic cord. Thus, the outer dimensions of the catheter 1 are relatively small and its distal end 2 also has good flexibility.

The use of bare conductors or wires or windings for forming the poles 3 allows either larger cross sections of these conductors or produces small outer dimensions and a bare conductor has greater flexibility than a conductor with an insulating coating.

Here, the individual windings of the multi-turn coil each extend parallel to each other, thus have matching dimensions and form a practically even coil on the outside.

1. Medical catheter (1) comprising several poles (3) or electrodes arranged axially one next to the other or one behind the other for stimulating and/or detecting potentials from an organ of the human body, a coil formed from several electrically conductive windings extending parallel to each other, wherein each of the windings leads to one of the poles (3) arranged at a distal end (2) of the catheter (1), the electrically conductive windings for forming the poles (3) are bare windings at the distal end of the catheter and insulation (4) is arranged between the bare windings at least in a region of the poles (3) and the insulation is formed by windings that are made from insulating material or by wound plastic cords and that extend between the bare windings.

2. Catheter according to claim 1, wherein for forming the poles (3) by the windings, at least a last turn of the winding at the distal end is left bare at least partially or across an entire periphery of the coil or the windings for forming the poles (3) are left bare across a fraction of a length or across an entire length of the coil.

3. Catheter according to claim 1, wherein the wound insulation (4) arranged between the electrically conductive windings has a cross-sectional shape that is round.

4. Catheter according to claim 1, wherein the windings are formed from at least one of wires, multi-strand wires, flat wires, or round wires and shaped into a multi-turn coil.

5. Catheter according to claim 1, wherein the windings of the multi-turn coil have matching dimensions and extend coaxially one next to the other.

6. Catheter according to claim 1, further comprising an outer, insulating coating (5) that extends up to the distal end (2) of the coil with the poles (3), and openings (7) are located in the outer, insulating coating in a region of the bare windings for forming small surface-area poles (3).

7. Catheter according to claim 1, wherein the windings are coated or covered in a direction toward a proximal end of the catheter (1) with insulation such that the poles (3) do not repeat on a meridian line of the coil in the direction toward the proximal end.

8. Catheter according to claim 6, wherein the coating (5) has only one of the openings (7) on the periphery in the region of the bare windings for forming each of the poles (3) and the openings (7) are arranged coaxially one next to the other.

9. Catheter according to claim 1, further comprising electrically conductive or metallic rings (8) arranged coaxially on an outside of the bare windings forming the poles (3) on the distal end (2), and insulation (9) is arranged between the conductive or metallic rings and the bare windings, and one of the windings forming one of the poles (3) contacts a respective one of the rings (8).

10. Catheter according to claim 1, wherein a number of the poles is increased via additional conductors (10) or wires guided in an inner lumen of the windings of the coil, past the distal end of the coil to a carrier (11) having elements that form additional poles (13) and that are each connected to one of the additional conductors (10).

11. Catheter according to claim 10, wherein the additional poles (13) are arranged one next to the other in an axial direction of the catheter (1) on the carrier (11).

12. Catheter according to claim 10, wherein the carrier (11) includes contact points arranged on an outside thereof or on peripheral rings that are bare at least in some regions and that are used as the additional poles (13) and are connected to the additional conductors (10) guided through the inner lumen of the coil that extend outward from a hollow inner space of the carrier (11).

13. Catheter according to claim 11, wherein at least one part or all of the additional conductors (10) guided through the inner lumen of the coil for the additional poles (13) provided at the distal end of the catheter (1) are insulated.

14. Catheter according to claim 11, wherein the distal end (2) having the poles (3, 13) has, at least across a part of its extent, a decreasing or tapering, conical, or rounded shape.

15. Catheter according to claim 11, wherein the inner lumen of the coil and the windings forming it have a cross section such that there is space for a stiletto or a mandrin or similar guide element in addition to the additional conductors (10) running therein.

16. Catheter according to claim 1, wherein the wound insulation (4) arranged between the electrically conductive windings has a cross-sectional shape that is adapted to a shape of intermediate spaces between the windings forming the poles (3).

17. Catheter according to claim 1, wherein an inner lumen of the coil and the windings forming it have a cross section such that there is space for a stiletto or a mandrin or similar guide element.
